(19) United States
(12) Patent Application Publication
(10) Pub. No.: US 2023/0000559 A1
(43) Pub. Date: Jan. 5, 2023

(54) SYSTEM & METHOD FOR MATCHING THE RESULTS OF A CT SCAN TO A NASAL-SINUS SURGERY PLAN TO TREAT MIGRAINE HEADACHES

(71) Applicant: KEVIN RAYNARD SMITH, HOUSTON, TX (US)

(72) Inventor: KEVIN RAYNARD SMITH, HOUSTON, TX (US)

(21) Appl. No.: 17/879,890

(22) Filed: Aug. 3, 2022

Related U.S. Application Data
(63) Continuation of application No. 15/628,623, filed on Jun. 20, 2017, now abandoned.
(60) Provisional application No. 62/352,473, filed on Jun. 20, 2016.
(60) Provisional application No. 62/352,475, filed on Jun. 20, 2016.

(51) Int. Cl. A61B 34/10; A61B 6/00; G06T 7/00; G06T 7/11; A61B 5/00; A61B 5/08

(52) U.S. Cl. CPC A61B 34/10; A61B 6/5217; G06T 7/0012; G06T 7/11; A61B 5/0037; A61B 5/0042; A61B 5/08; A61B 5/4824; A61B 5/4836; A61B 5/4887; A61B 2034/101; A61B 2090/3762

(57) ABSTRACT

A method and system to treat headaches in a patient by performing surgery via at least one nostril. Data from a computer tomography scan of at least one nasal cavity and one sinus cavity of the patient and a completed headache questionnaire are matched to at least one nasal/sinus surgery plan to operate on at least one of: a nasal septum, at least one sinus cavity and at least one turbinate of the patient. The surgery plan is executed by installing a topical local anesthetic and decongestant onto the at least one turbinate forming an anesthetized decongested nasal cavity; infusing an anesthetic into the anesthetized decongested nasal cavity of the patient; dilating the at least one sinus ostium; incising at least one of: a first mucosal flap or a second mucosal flap of the nasal septum of the anesthetized decongested nasal cavity to expose deviated septal cartilage and bone; removing deviated cartilage and/or bone of the nasal septum; fracturing the at least one turbinate laterally away from the nasal septum; inspecting between the first mucosal flap and the second mucosal flap for a residual broken bone, a residual segment of cartilage or combinations thereof, surgically closing the first mucosal flap and the second mucosal flap of the nasal septum; and suctioning unwanted matter from the anesthetized decongested nasal cavity. An interactive system guides the surgery and provides a record thereof.

### TECHNICAL FIELD OF THE INVENTION

The present invention generally relates to the treatment of migraine headaches and, more particularly, to systems and methods for the treatment of headaches by matching computer tomography (CT) scan and other patient data to a treatment plan involving nasal and/or sinus (nasal/sinus) surgery.

### BACKGROUND OF THE INVENTION

Historically, a wide assortment of potential etiologic factors were identified for migraine development, including vasodilation and vasoconstriction of extra- and intracranial vessels, cerebral anoxia, thrombocyte aggregation, serotonin levels, and prolactin levels. Specific migraine triggers include rapid changes in the weather, alcohol, caffeine, cheese, fever, lack of sleep, stress, and menstrual periods. With this range of potential causes, there was no clear biological marker for migraine, and diagnosis was dependent on assessment of numerous clinical criteria having to do with duration, location, quality of pain, and concurrent development of other symptoms, such as nausea and/or vomiting, photophobia, and phonophobia.

In a typical treatment scenario for suspected migraine, patients begin by using over-the-counter medication. If this does not bring sufficient relief, they proceed to a primary care physician, who may prescribe non-steroidal anti-inflammatory medications or, if necessary, narcotics. Ultimately, the patient may be referred to a neurologist who may seek a computer tomography (CT) scan or MRI of the brain. Ninety-five percent of all brain scans performed for migraine patients are normal. Therefore, physicians begin prescribing migraine-specific medications and/or ultimately referring the patient into chronic pain management.

According to some studies, migraine headaches are diagnosed in approximately 18% of women and 6% of men, affecting as many as 40 million people per year in the United States. These debilitating headaches present a crippling personal and economic burden, not least because historically their etiology has been complex and the most effective way to diagnose and treat them is uncertain. In part because of this uncertainty, more than half of migraine sufferers have not been formally diagnosed, nor have they ever received prescription medications.

One study showed 43% of migraine sufferers reported five or more days of headache pain in the previous three months. More than half reported that their pain forced them to go to bed for days at a time, and the same percentage reported a reduction of 50% or more in work or school productivity. Nearly 25% had sought care in an emergency room or urgent care clinic.

Another study reported that families with at least one person suffering from migraines had total annual medical costs that were 70% higher than families without migraine. In those families with an adult sufferer, this amounted to about $4,700 per year. Interestingly, a significant proportion of these costs was not for direct treatment of headache symptoms, but was related to comorbid conditions and to treatment delivered to spouses and family members. For example, the spouse of a migraine sufferer had total healthcare costs that were 24% higher than a counterpart in a non-migraine family, and a child had costs that were 11% higher.

One of the “hidden” costs of migraine is the loss of productivity in employees who come to work but cannot perform at optimum levels. Researchers from the University of Michigan conducted a study with Chicago-based Bank One to determine the costs in lost productivity in its workers who suffered from migraine. Health risk appraisal questionnaires were completed by 19,853 employees, of whom 20% reported a history of migraine headache. Responses were combined with demographic and payroll data to estimate the corporate costs due to migraine-related absenteeism and reduced productivity. While direct costs due to absenteeism totaled $21.5 million, estimated costs due to lost productivity were even higher, at $24.4 million. The magnitude of these costs reflects the fact that the prevalence of migraines peaks during the ages of 25 to 55 years, the prime working years. The surprising conclusion of this study was that productivity costs related to chronic diseases such as migraine, arthritis, allergies, and back pain, are equivalent to the medical costs related to severe conditions such as heart disease and cancer.

Another set of costs related to migraine reflects the difficulty of diagnosis in patients suffering from severe headaches. In the one year period from February 2003-February 2004, 689 patients with a primary diagnosis of headache NOS (not otherwise specified) were admitted to a major teaching hospital in Houston, Tex. The average length of stay for these patients was 3.9 days, with a total billed cost for all patients of nearly $14 million for a single one-year period.

Therefore, a longstanding need exists for effective methods and systems to permanently reduce the frequency or severity of or completely eliminate migraine headaches by performing a surgery to eliminate the root cause.

The medical systems and methods of the present invention meets such needs.

### SUMMARY OF INVENTION

The present invention is directed to specific systems and methods which surgically treat persistent headaches such as migraine headaches based on matching a computer tomography (CT) scan of the patient's sinus cavities with a nasal-sinus surgical treatment plan aimed at optimally reducing or eliminating the root cause of persistent headaches. The present inventor has discovered that the activation of the trigeminal nerve by contact with hypertrophied turbinates and the rigid confines of the nasal cavity (even without a deviated septum) can cause a headache. More commonly, a septal deviation or septal spur impinges on the turbinates causing a range of headaches from mild intermittent or severe daily pain that is commonly diagnosed as migraine. A septal deviation is defined as an off-center deflection of the septum from a line extending from the crista galli to the maxillary crest. A septal spur is a severe, sharp deflection of the septum that can pierce the turbinates or lateral wall of the nose.

The present system and methods may be used on patients initially presented with migraine headache in addition to chronic nasal congestion and/or recurrent sinus infections. Such patients are initially manually examined with the application of a topical decongestant (e.g. Phelylephrine) and anesthetic (e.g. Pontacaine® or Tetracaine®) spray within the nose in order to obtain a clear view of the entire nasal airway with an endoscope. Septal deviation and/or septal spurs are commonly diagnosed using this procedure. After application of the spray, patients with a headache are observed to see if there was a reduction/resolution of the pain, as further evidence of a rhinogenic cause. (To prevent introducing bias, patients may be told the are receiving only a decongestant spray, then questioned at the end of the visit to see if there was a reduction or resolution of their headache). As an alternative test for rhinogenic etiology of the headaches, some patients were sent home with Afrin® decongestant nasal spray and instructed to use it at the onset of a headache to see if it inhibited or diminished the headache. In either case, these are not conclusive tests but simply additional data gathering as the spray may not reach the area(s) of concern.

Each patient receives a CT scan of their sinuses to evaluate the degree and quantity of septal deviations and to assess other possible nasal/sinus abnormalities (e.g., cyst, polyp, osteoma, mucosal thickening, scar, sinusitis, turbinate hypertrophy, concha bullosa). Although endoscopic exams are frequently used to evaluate patients for a deviated septum, the present invention uses CT scans to assess subtle contact points that may not be visible by nasal endoscopy, and to thoroughly examine nasal and sinus areas that are generally not accessible using nasal endoscopic technology, e.g. the sinuses and superior turbinates, particularly for patients who have been previously diagnosed with migraine headaches.

Patients with a diagnosis of suspected migraine headaches in addition to septal deviation and/or septal spurs are asked to complete a pre-treatment headache questionnaire to provide the system and method of the present invention with data regarding:
How long the patient has suffered from headaches (weeks, months, years)
Frequency (number per week or month, standardized to number per month for analysis)
Location (Typical location for rhinogenic headaches include: forehead, temples, periorbital, cheek, top or back of head, perinasal)
Severity of pain (minimum, maximum, and average, on a scale of 1 to 10)
Quality of pain (e.g., constant, pounding, etc.)
Time of day or night of headache pain onset
Triggers that cause the headaches
Occurrence of sinus infections
Occurrence of hypertension
Family history of headaches
History of nasal or facial trauma
Amount of nasal congestion from 0.1 percent to 100 percent
Previous treatment history for headaches
Prior MRI or CT scan of the brain

Patients are considered to have a diagnosis of migraine headache if they had been previously diagnosed with migraine by a medical doctor such as an internist (primary care doctor), neurologist, or ear, nose, and throat (ENT) specialist prior to presentation or if their description of headache symptoms (duration, frequency, severity, location, occurrence with nausea/vomiting and/or photophobia and phonophobia) is consistent with migraine classification criteria from the International Headache Society.

The method and system of the present invention applies an algorithm using the CT scan and the patient supplied headache data as inputs and which matches the input data with a specific nasal and/or sinus surgical headache treatment plan. In accordance with one preferred embodiment, the surgical plan with the highest number of matches is selected as a proposed treatment plan. The surgeon subsequently independently validates the proposed treatment plan based upon a compilation of the physical examination, review of the CT scan and assessment of the patient's medical information including the headache data.

If the surgeon is satisfied with the proposed headache treatment plan, the patient is treated with the selected surgical plan. The goal of the definitive plan is to eliminate any and all contact between the septum and nasal soft tissues including the turbinates and/or eliminating any sinus abnormalities. The surgical plan may include: (a) correcting the septal abnormality(s) by (1) fracturing, shaving or removing septal bone, (2) fracturing, shaving or removing septal cartilage (b) correcting turbinate abnormalities by reducing, fracturing, ablating, cauterizing, partially or totally excising one or more turbinates (superior, middle, or inferior) to eliminate contact points with the septum and/or decrease nasal congestion and (c) lysing nasal scar tissue, and/or (d) correcting sinus abnormalities by utilizing balloon sinuplasty or traditional endoscopic sinus surgery when indicated by the algorithm to (1) enlarge at least one ostium, (2) removing at least one polyp, cyst, osteoma, (3) remove sinus mucosa, bone, pus, (4) performing a lavage on at least one sinus.

One study assigned a dollar value of $5,375 per migraine sufferer per year for absenteeism and $6,100 per migraine sufferer per year for loss of productivity. This is in addition to the increased total medical costs of $4,700 per year in families with an adult migraine sufferer.

Based upon the inventor's own clinical results, the reduction or elimination of migraines via the systems and methods of the present invention are estimated to result in a total savings per year of $10.8 billion in absenteeism, $12.2 billion in lost productivity, and $9.4 billion in medical costs. The cost of providing CT scans for all patients with suspected migraine may be recouped in the first year alone.

Given the magnitude of the potential benefit, both in terms of relief of patient suffering and of reduced healthcare and business costs, the headache treatment systems and methods of the present invention implement a CT scan of the nose and sinuses and matches the finding of the CT scan ((1) nasal abnormalities such as a deviated septum, septal spurs, and/or any contact points between the septum and turbinates or between the turbinates and other rigid nasal structures or (2) sinus abnormalities such as narrow or obstructed ostium, polyp, cyst, mucosal thickening, sinusitis, osteoma) to the patient supplied data to create a nasal and/or sinus surgical treatment plan. Those patients whose CT scan does not match a surgical headache treatment plan may be referred to other specialists. The systems and methods of the present invention identifies and surgically treats those patients with headaches stemming from rhinogenic triggers, providing fast and lasting relief in a cost-effective way.

### BRIEF DESCRIPTION OF DRAWINGS

The detailed description will be better understood in conjunction with the accompanying drawings in which:

FIG. 1 depicts a front view of a congested sinus with a bone spur according to one or more embodiments.

FIG. 2 is a front view of a head of a patient showing sinus cavities according to one or more embodiments.

FIG. 3 is a side view of nasal cavities of a patient according to one or more embodiments.

FIG. 4 depicts a computer tomography scan of congested nasal and sinus cavities according to one or more embodiments.

FIG. 5 depicts a computer tomography scan of anesthetized decongested nasal cavities according to one or more embodiments.

FIG. 6 depicts a kit with equipment used to implement migraine reduction according to one or more embodiments.

FIG. 7 depicts a data storage according to one or more embodiments.

FIGS. 8A and 8B depict a method to use the kit to reduce migraine headaches in a patient according to one or more embodiments.

The present embodiments are detailed below with reference to the listed Figures.

### DETAILED DESCRIPTION OF PREFERRED EMBODIMENTS

Before explaining in detail the preferred embodiments of the present invention, it is to be understood that the invention is not strictly limited to the described embodiments but may be practiced with various modifications or further improvements thereto.

The preferred embodiments disclose a method and system of treating headaches using patient obtained data and the results of a CT scan of the nasal cavity and at least one sinus cavity of the patient to identify specific structural abnormalities and to match the patient obtained data and identified structural abnormalities to a specific nasal-sinus surgical treatment plan for implementation by a qualified surgeon. The structural abnormalities identified from the CT scan may include one or more of the following: a deviated septum; a septal spur; turbinate hypertrophy; concha bullosa of the middle, superior and/or supreme turbinate; disease of the frontal, maxillary, and/or sphenoid sinuses including mucosal thickening, polyp, cyst, complete opacification, osteoma, narrowing or obstruction of ostium including the recess of the frontal sinus; ethmoid sinus mucosal thickening or complete opacification of either the anterior, posterior or the entire ethmoid sinus.

The method and system of the present invention implements a decision tree to surgically treat recurrent headaches based upon the patient obtained data and the results of the CT scan to identify a proposed nasal/sinus surgical treatment plan that can be implemented to reduce or eliminate the frequency or severity of the headaches.

The method and system utilize a computer tomography scanner (CT scanner) configured to perform a scan on the nasal cavity and at least one sinus cavity of a patient. A processor is connected to data storage that stores the results of a computer tomography scan which may indicate one or more of the following: a deviated septum; a septal spur; turbinate hypertrophy; concha bullosa of the middle, superior and/or supreme turbinate; disease of the frontal, maxillary, and/or sphenoid sinuses including mucosal thickening, polyp, cyst, complete opacification, osteoma, narrowing or obstruction of ostium including the recess of the frontal sinus; ethmoid sinus mucosal thickening or complete opacification of either the anterior, posterior or the entire ethmoid sinus.

The data storage also includes patient headache data acquired from plurality of answers of the headache questionnaire to identify one or more of the following:
How long the patient has suffered from headaches (weeks, months, years)
Frequency (number per week or month, standardized to number per month for analysis)
Location (Typical location for rhinogenic headaches include: forehead, temples, periorbital, cheek, top or back of head, perinasal)
Severity of pain (minimum, maximum, and average, on a scale of 1 to 10)
Quality of pain (e.g., constant, pounding, etc.)
Time of day of pain
Triggers that cause the headaches
Occurrence of sinus infections
Occurrence of hypertension
Family history of headaches
History of nasal or facial trauma
Amount of nasal congestion from 0.1 percent to 100 percent
Previous treatment history for headaches
Prior MRI or CT scan of the brain

The data storage also includes a plurality of proposed medical treatment plans to surgically treat at least one of: a nasal septum of a patient, at least one sinus cavity of a patient and at least one turbinate of a patient.

The data storage also includes computer instructions to match the data obtained from CT scan and patient headache data to at least one of the proposed nasal/sinus surgery treatment plans, compare the matches, and identify the proposed nasal/sinus surgery treatment plans with the greatest number of matches as the nasal/sinus surgery treatment plan to implement.

The computer instructions compare the patient's actual scan data with model patient data to identify deviations in the patient's scan data from the model data. For example, the computer instructions may assign numeric values to characteristics of the patient's nasal and sinus structures captured in the patient's scan and compare these values with baseline values for model patient data scaled for the patient's physical size and weight to determine whether the scan data indicates one or more of the following: a deviated septum; a septal spur; turbinate hypertrophy; concha bullosa of the middle, superior and/or supreme turbinate; disease of the frontal, maxillary, and/or sphenoid sinuses including mucosal thickening, polyp, cyst, complete opacification, osteoma, narrowing or obstruction of ostium including the recess of the frontal sinus; ethmoid sinus mucosal thickening or complete opacification of either the anterior, posterior or the entire ethmoid sinus.

The computer instructions may match computer tomography scan values showing a deviation of the septum with a maxillary crest and patient data indicating nasal impairment with one of the following treatment plans: (1) to remove the maxillary crest bone with an osteotome; (2) endoscopic removal for an isolated abnormality by making an incision to the mucosa anterior to the deflection; and (3) removal of deflected bone and cartilage within the maxillary crest.

The computer instructions may match computer tomography scan values showing a mild deviation of the cartilage without contact to the lateral nasal wall and a given set of patient headache data values with a treatment plan to optionally remove the mild deviation if causing nasal impairment in breathing.

The computer instructions may match computer tomography scan values showing a moderate deviation of the cartilage and a given set of patient headache data with a treatment plan to optionally remove the moderate deviation leaving behind at least 1 cm of cartilage dorsally and caudally to maintain nasal tip and dorsal support.

The computer instructions may match computer tomography scan values showing a caudal cartilage deviation and a given set of patient headache data values including breathing impairment with a treatment plan to remove and reconstruct the cartilage with a straight piece of septal cartilage, or implant and to splint with nasal splints for 7-14 days.

The computer instructions may match computer tomography scan values showing a a mild deviation of the bony septum and a given set of patient headache data with (b) one of the following treatment plans (1) fracture the deviation to the midline; (2) remove the deviation endoscopically or with traditional septoplasty based upon which procedure will result in appropriate bone removal, particularly superiorly, posteriorly to the keel of the sphenoid and inferiorly to minimize the possibility of leaving any bone that can contact the turbinates.

The computer instructions may match computer tomography scan values showing isolated septal spurs and a given set of patient headache data values with a treatment plan to endoscopically remove the isolated spurs by making an incision anterior to the deflection, elevating the mucosa off both sides of the bone and removing the bone, suturing the mucosal flaps with dissolvable suture or splinting them with bilateral sponge packing whereby for small spurs the proposed surgical treatment plan may allow the mucosa to be simply laid back in place to heal.

The computer instructions may match computer tomography scan values showing opacification or mucosal thickening of the frontal sinus and a given set of patient headache data values indicating pressure headaches in the forehead with a treatment plan for frontal balloon sinuplasty with lavage.

The computer instructions may match computer tomography scan values showing osteoma of the frontal sinus and a given set of patient headache data values with a treatment plan to remove the osteoma with the traditional sinus technique of using forceps or other appropriate instrument if the osteoma is located in the recess cells and removing any osteoma if located higher in the recess or sinus only if symptoms persist in the forehead.

The computer instructions may match computer tomography scan values showing opacification, mucosal thickening or a narrow or obstructed outflow tract or ostium of the maxillary sinus and a given set of patient headache data values with a treatment plan for maxillary sinus ostium enlargement with balloon sinuplasty or other tool or method, and may include lavage.

The computer instructions may match computer tomography scan values showing at least moderately sized cysts in the sinus and a given set of patient headache data with a treatment plan for cysts or mucoceles by using sinus forceps to remove the mucosa or by rupturing them with a suction or other tools designed to do such.

The computer instructions may match computer tomography scan values showing opacification, mucosal thickening or a narrow or obstructed outflow tract or ostium of the sphenoid sinus and a given set of patient headache data values with (b) a treatment plan for sphenoid sinus ostium enlargement with a balloon sinuplasty or other tool or method and may include lavage.

The computer instructions may match computer tomography scan values showing anterior opacification, mucosal thickening or osteoma of the ethmoid sinus and a given set of patient headache data values with a treatment plan for an anterior ethmoidectomy using an endoscope, sinus forceps, shaver or other tools designed for such treatment.

The computer instructions may match computer tomography scan values showing complete opacification, mucosal thickening or osteoma of the total or posterior ethmoid sinus and a given set of patient headache data values with a treatment plan for a total or posterior ethmoidectomy using an endoscope, sinus forceps, shaver or other tool designed for such treatment.

The computer instructions may match computer tomography scan values showing the inferior turbinates in contact with the septum and patient headache data values indicating no significant nasal airway impairment experienced by the patient with a surgical treatment plan to outfracture the inferior turbinates away from the septum to eliminate the contact between the two.

The computer instructions may match computer tomography scan values showing the inferior turbinates are in contact with the septum and patient headache data values indicating mild to moderate nasal airway impairment experienced by the patient (less than or equal to 50% of the time) with a surgical treatment plan to outfracture the inferior turbinates away from the septum with ablation of the turbinate tissue to conservatively reduce their size.

The computer instructions may match computer tomography scan values showing the inferior turbinates are moderately enlarged and are in contact with the septum, nasal floor or lateral nasal wall, and a given set of patient headache data values indicating moderate to severe nasal airway impairment experienced by the patient (more than 50% of the time) with a surgical treatment plan to outfracture the inferior turbinates away from the septum and to excise and cauterize ¼-⅓ of the inferior portion from front to the tail angling downward posteriorly to avoid excision of the tail where the feeding vessels diameter is the largest and difficult to cauterize to control hemostasis.

The computer instructions may match computer tomography scan values showing the patient's middle turbinates with mild hypertrophy with close approximation to the septum and a given set of patient headache data values indicating mild perinasal pain with a treatment plan for surgical treatment to reduce their width with forceps or other tool designed to do such.

The computer instructions may match computer tomography scan values showing the patient's middle turbinates with moderate hypertrophy and close approximation to or contact with the septum and patient headache data values indicating perinasal pain at the nasal bones with at least one of the following treatment plans: (a) to reduce their width with forceps or other tool designed to do such or (b) to partially excise the inferior 2-8 mm margin from front to back angling the inferior at the tail to avoid the large feeding vessels that are difficult to cauterize to control hemostasis; (c) to gently outfracture away from the septum with an elevator.

The computer instructions may match computer tomography scan values showing the patient's middle turbinates with moderate to severe hypertrophy and contact with the septum and patient data values indicating perinasal pain at the nasal bones with a treatment plan to partially excise the inferior 2-8 mm margin and cautery of the remnant.

The computer instructions may match computer tomography scan values showing small to medium concha bullosa having minimal contact with the septum and a given set of patient headache data values with a treatment plan to reduce its width by squeezing the turbinate with forceps or another tool designed for such.

The computer instructions may match computer tomography scan values indicating medium to large concha bullosa having substantial contact with the septum and a given set of patient headache data values with a treatment plan to excise its lateral portion and cauterize the edges of the remnant.

The computer instructions may match computer tomography scan values indicating the superior turbinates are in contact with the septum, regardless of the size or presence of concha bullosa, and a given set of patient headache data values, with a surgical treatment plan for outfracturing the contacting turbinates away from the septum with an elevator or other tool without traumatizing the mucosa to avoid injury to the olfactory nerve fibers and impair the sense of smell or to perform sphenoid balloon sinuplasty by pulling the balloon anterior to the sinus ostium and insufflating.

The computer instructions may match computer tomography scan values indicating the supreme turbinates (1) are in contact with the septum, regardless of the size or presence of concha bullosa, and a given set of patient headache data values, with a surgical treatment to perform a gentle outfracture of the contacting supreme turbinates with an elevator, balloon or other tool designed for such taking care to perform the outfracture gently without traumatizing the mucosa to avoid injury to the olfactory nerve fibers and impair the sense of smell.

The system may also include stored instructions detailing the steps of implementing the process as well as programming to interactively display such instructions. The system may optionally be programmed to provide a check list for the procedure wherein a user input must be received and stored indicating each step has been completed in the checklist or otherwise providing visual indication of what structures need to be operated upon and may provide visual indication when the procedure for a given structure has been completed. Such instructions may include one or more of the following steps:
computer tomography scan review;
correlating the highest matching surgical treatment plan to alleviate migraine headaches identified by the migraine/headache algorithm (computer tomography scan nasal/sinus findings, patient questionnaire data) to an endoscopic examination of the patient;
reviewing the plan with the patient;
obtaining patient consent;
providing pre-operative instructions including getting medical clearance when necessary, stopping all anti-coagulants such as certain medications, herbal/nutritional supplements and alcohol along with caffeine, tobacco and other medication within a certain time period prior to the procedure.
post-operative instructions which may include (a) for the patient to acquire the appropriate medications which may include one or more of the following (1) over-the-counter medications or similar medication: decongestant nose spray, steroid nose spray, mucolytic, saline nose spray, antibiotic ointment; and (2) which may additionally include the following prescription medicines: pain medication, antibiotic, low dose steroid pack, and/or anti-nausea medication, (3) and may additionally include the following compounding pharmacy solution for nasal irrigation comprised of one or more of the following: antibiotic, antifungal, anti-inflammatory medication (b) elevation of the head at least 30 degrees above the horizontal plane or supine position, (c) application of cold compresses to the forehead, nose and cheeks, (d) no strenuous activity for 3 weeks.

These instructions may include instructions for the patient and anesthesia team to meet, obtain anesthesia consent, start an IV, review the anesthesia plan.

The instructions may include in pre-operative instructions such as decongesting each nostril to be examined or operated on such as by applying one or two puffs of decongestant spray in each nostril of the patient to be decongested or spraying each nostril with a mixture of topical anesthetic and decongestant such as Tetracaine 4% and Phenylepherine 1% mixed at a ratio of 1:1 for a final percentage Tetracaine 2% and Phenylephrine 0.5%.

The instructions may include manually reviewing the CT scan noting specific and unusual anatomy, i.e. aplastic or small sinuses, cribriform, and contact between septum and turbinates.

The instructions may include placing pledgets such as cotton soaked in decongestant or the tetracaine/phenylephrine mixture for approximately two minutes over the inferior turbinate and more pledgets higher in the nose medial to the middle turbinate.

The instructions may include performing a nasal endoscopy for nasal abnormalities.

The instructions may also emphasize taking note of the potential of future injury causing one or more deflection or septal spur that may produce a headache.

The instructions may include details on performing Balloon Sinuplasty or traditional method for enlarging the ostium for all indicated sinuses to improve drainage, reduce infection rates and relieve barometric pressure headaches that trigger migraine. The instructions may include gently fracturing the indicated middle turbinate to gain access to the respective sinus ostium. The instructions may provide the sequence of approaching the sinuses in this order: frontal left then right, maxillary left then right, then sphenoid left then right. The instructions may include performing a lavage in one, several or all sinuses with normal saline with or without an antibiotic and steroid regardless of their appearance on the computer tomography scan to clear current infection, pus or mucus and to reduce the amount of blood that may collect in the sinus at the time of surgery and reduce the time for the sinus to clear and minimize infection.

Where balloon sinuplasty of a sphenoid sinus ostium is indicated, the instructions for ballooning the ostium of the sphenoid sinus may include confirming the placement in the sinus ostium by gently tugging the balloon when fully inflated to ensure firm resistance with no movement of the balloon and a warning that if the balloon moves outward from the sphenoid area when fully inflated then it is not in the sinus ostium.

The instructions may also include a warning that attempts should be repeated until cannulation of the ostium is achieved and may provide an exception for a small sphenoid bud or a sphenoid sinus ostium that has been previously ballooned or inherently large. The instructions may include ballooning to enlarge the sphenoid outflow tract by pulling the balloon anterior to the ostium and insuflating.

When an ethmoidectomy (defined as removing the compartments of bone and mucosa within either part of or the entire ethmoid sinus cavity usually performed to eliminate the frequency of sinus infections and/or eliminating pressure and pain) is identified as part of the headache treatment plan (based on the findings of mucosal thickening on the CT scan) along with patient data indicating headache pain between or behind the eyes, the instructions may include:
(a) using sinus forceps or other tool designed for such to enter into and remove the ethmoid bulla and remove the anterior and posterior cells using a tool such as a curette or suction to enter compartments of the ethmoid sinus;
(b) starting the dissection on the inferior part of the ethmoid bulla and staying low throughout the cavity to avoid violation of the fovea ethmoidalis superiorly; and
(c) alternating a curette and sinus forceps or other tool designed for such to define the medial, lateral, superior, inferior and posterior extent of the ethmoid cavity;
(d) reiterating that the final cavity of the ethmoid sinus should resemble a ‘cave’ in most cases with an arched roof of the fovea ethmoidalis, the lateral wall of the lamina paprycea, the medial border of the middle turbinate and posteriorly the face of the sphenoid sinus;
(e) measuring the extent of ethmoid sinus dissection (1) from the posterior most extent of a total ethmoid dissection to the edge of the nostril or nasal sill, providing the following approximate measurement guide: Teens and young adults 5.5-6.0 cm; Adult women 6.0-6.5 cm; and Adult men 6.5-7.0 cm, and (2) from the posterior most extent of an anterior ethmoid dissection to the edge of the nostril or nasal sill which is any measurement less than a total ethmoidectomy.

The instructions may include (1) injecting into the ethmoid cavity a water-based antibiotic ointment such as Mupirocin administered with a syringe such as a 3 cc syringe attached to a needle such as an 18 gauge or angiocath; (2) trimming a PosiSep or a similar expandable dissolvable sponge dressing to size and placing it in the ethmoid defect; (3) expanding the sponge dressing with a solution such as a solution comprised of normal saline, steroid and antibiotic.

The matched headache treatment nasal/sinus surgery plan based upon a CT scan showing abnormalities of the middle turbinates may be one of several options depending on their size and close proximity to the septum. When the identified surgical treatment plan for the elimination of the headache involves: (1) gently outfracturing away from the septum, (2) reducing or (3) partially excising the middle turbinates to avoid contact with the septum, the instructions may include performing an outfracture of the turbinate using an elevator such as a Freer elevator or other tool designed for such, the reduction of the turbinate width with a straight hemostat or duckbill forceps or other tool designed for such, gently squeezing the head and inferior bulbous portion of the middle turbinate. Where the CT scan indicates large bulbous or extremely enlarged turbinates, the instructions for the indicated surgical plan may include excising the lower third of the turbinate using turbinate scissors (leaving behind the upper ⅔) and cauterizing the remaining margins.

The instructions for excision of a middle turbinate concha bullosa may include: (1) using a knife blade to enter along a vertical or sagital plane along the head of the turbinate, completing the excision with turbinate scissors and removing the lateral portion of the ‘egg shell’ with forceps or other tools designed for such; (2) gently cauterizing the margins of the remaining turbinate to control hemostasis.

The matched headache treatment surgery plan based upon a CT scan showing abnormalities of the inferior turbinates may be one of several options depending on the degree of nasal obstruction and their appearance on the CT scan. The matched plan may be performing an outfracture where the patient data indicates no perceived obstruction by the patient. The matched plan may be performing an outfracture with ablation where the patient data indicates nasal obstruction 50% of the time or less and where the CT scan shows turbinates of normal or mild hypertrophy. Where the CT scan indicates turbinates that touch the normal straight septum or the normal confines of the nasal cavity (floor or lateral nasal wall), the matched surgical plan may be excising them just enough to eliminate the contact.

The instructions for excision of a middle turbinate concha bullosa may include: (1) using a knife blade to enter along a vertical or sagittal plane along the head of the turbinate, completing the excision with turbinate scissors and removing the lateral portion of the ‘egg shell’ with forceps or other tools designed for such; (2) gently cauterizing the margins of the remaining turbinate to control hemostasis.

However, where the patient data indicates one or more of nasal congestion greater than 50% of the time, nasal obstruction that occurs most mornings or nights, persistent mouth breathing or breathing impairment that impedes or limits exercise, the matching surgical plan may be a turbinectomy. For such a plan, the instructions may include excising the lower ¼-⅓ of the turbinate with scissors and cauterizing the remnant with a suction cautery or other tools designed for such. The instructions may include angling the scissors downward upon excision near the tail of the turbinate to avoid transecting the larger feeding vessels that can retract into the lateral nasal wall making it difficult to control hemostasis.

The matching headache treatment surgical plan when middle turbinate contact with the septum is seen on the CT scan, may include instructions to gently outfracture the turbinate with an elevator or other tool designed for such to remove all contact. The instructions may include a warning to avoid creating tears of the mucosa to prevent scar formation.

The headache treatment surgical plan matching a CT scan showing a superior turbinate (with or without a concha bullosa) making contact with the upper perpendicular plate of the ethmoid it may include instructions to gently outfracture the turbinate away from the septum with an elevator or other tool designed for such. The instructions may include a warning to avoid lacerating or otherwise damaging the superior turbinate to prevent damage to the olfactory nerve fibers which has branches covering the turbinate.

The instructions may also include placing PosiSep or similar dissolvable sponge type packing and may include expanding the sponge with a solution comprised of normal saline, steroid and antibiotic when tears are encountered with the septal mucosa in the region of the middle or superior turbinate to prevent scar band formation.

The instructions may include frequently suctioning the posterior nasopharynx throughout the procedure to prevent build-up of blood or clots that can be aspirated upon removal of the LMA or ET tube.

The instructions may include administering a topical anesthetic such as 2.0 ml of lidocaine jelly using a syringe such as a 3 ml and a needle such as an 18 gauge or angiocath within each nasal cavity to cover the septal mucosa and turbinates to aid in immediate post-operative pain control. The instructions may include allowing the jelly to sit for 1 minute or so and suctioning the excess. The instructions may include applying a drip pad underneath at least one nostril to collect bodily fluid drainage.

Tools used in the implementing of the identified nasal/sinus surgical procedure to treat the recurrent headache may include:
(a) an atomizer with a pump, which contains a topical local anesthetic and decongestant that when sprayed into the nose forms a decongested nasal cavity;
(b) an illumination device, such as a light emitting diode (LED) with a power supply with connections for a monitor, light cord and camera configured with an endoscope to enable inspection of the decongested nasal cavity and confirm the medical treatment plan identified as the best match and to inspect between mucosal flaps for residual septal bone fragment, residual fragment of cartilage or combinations;
(c) a sinus endoscope with a light cord and camera attached;
(d) a sinus dilation apparatus with insufflation device configured to perform dilation of at least one sinus ostium to undertake the matching medical treatment plan;
(e) a speculum and a pair of nostril retraction devices configured to expose at least one mucosal flap of a nasal septum;
(f) a plurality of saturation devices such as cottonoids or the like;
(g) a topical local anesthetic and decongestant used to saturate the saturation devices used to contact each of the plurality of turbinates of the patient to decongest the plurality of turbinates; and
(h) a syringe with a needle to infuse an anesthetic into a nasal cavity of the patient.

Other tools may include: a blade, an elevator, scissors, a hemostat, forceps, an osteotome, a mallet, a rongeur, and a nasal splint. These other tools may be used to perform one or more of: removing bone and cartilage of the nasal septum; reconstructing cartilage of the nasal septum; repositioning at least one of: bone and cartilage of the nasal septum; at least partially removing at least one ethmoid sinus of the patient; and opening at least one maxillary sinus ostium of the patient. Sutures or staples may be used to surgically close the exposed mucosal flaps of the nasal septum. A suction device can be used to remove unwanted bodily fluid from the decongested nasal cavity. A drip pad can be positioned underneath at least one nostril of the patient to collect bodily fluids.

The embodiments can help provide relief to patients that experience chronic migraine headaches. The system when implemented has been demonstrated to reduce migraine headaches in a given patient having a computer tomography scan indicating a surgical intervention by at least 50 percent.

The embodiments can provide an improved quality of life because the patient is able to manage the pain. The embodiments allow patients, who were once disabled, to return to work while lessening the pain and decreasing the number of headaches and migraine pain. The embodiments allow patients to have long term goals, such as going back to school, and taking care of their families, because they are no longer disabled from the powerful negative effects of chronic migraine headaches.

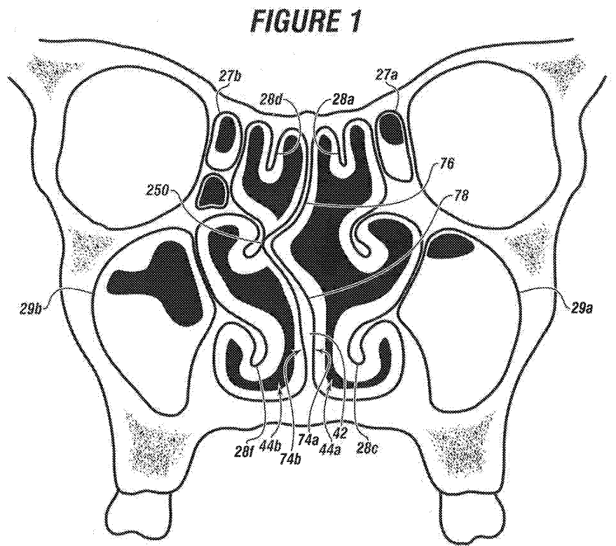

Turning now to the Figures, FIG. 1 depicts a front view of a congested sinus with a bone spur according to one or more embodiments. The patient can have at least one turbinate 28a-28f, a septal bone spur 250 that needs to be treated, mucosal flaps 74a and 74b, a residual broken bone 76, a residual segment of cartilage 78, a pair of ethmoid sinuses 27a and 27b, at least one maxillary sinus 29a and 29b, a nasal septum 42, nasal cavities 44a and 44b, or combinations thereof.

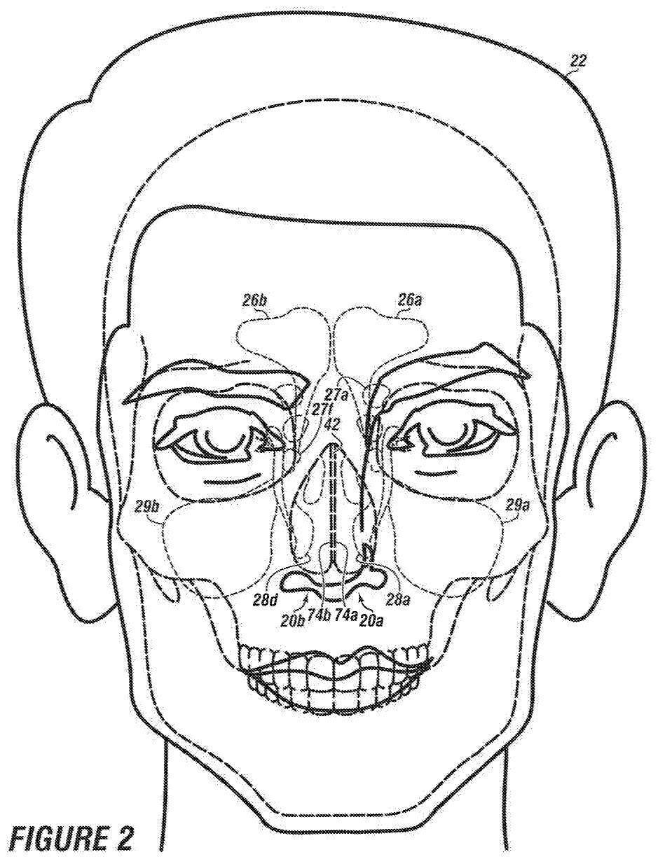

FIG. 2 is a front view of a head of a patient showing sinus cavities according to one or more embodiments.

In embodiments, the patient 22 can have a first mucosal flap 74a and a second mucosal flap 74b of a nasal septum 42, at least one sinus cavity 26a and 26b, a pair of ethmoid sinuses 27a and 27f, at least one turbinate 28a and 28d, and at least one maxillary sinus 29a and 29b.

The treatment of the headaches, namely migraine headaches, can be accessed through at least one nostril 20a and 20b.

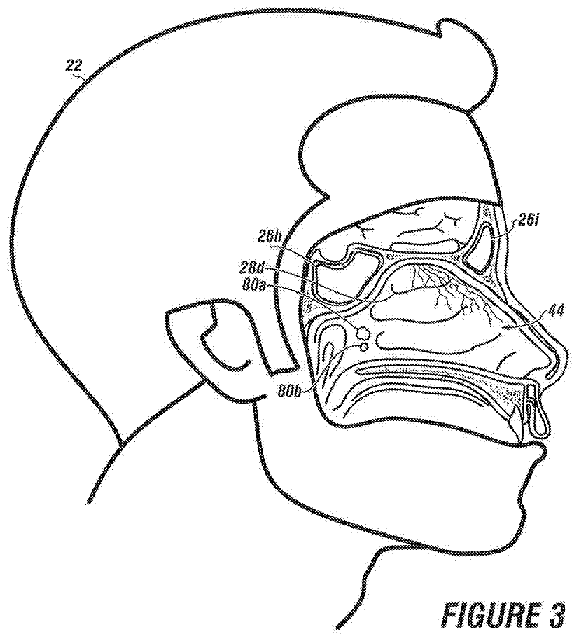

FIG. 3 is a side view of nasal cavities of a patient according to one or more embodiments.

In embodiments, the patient 22 can have at least one sinus cavity, such as sinus cavity 26h, a frontal sinus cavity 26i and at least one turbinate 28d in the nasal cavity 44. In this embodiment, the nasal cavity 44 is shown with unwanted matter 80a and 80b. The unwanted matter may include bodily fluids, bone, cartilage, injected fluids, man-made materials, human tissues, or combinations thereof.

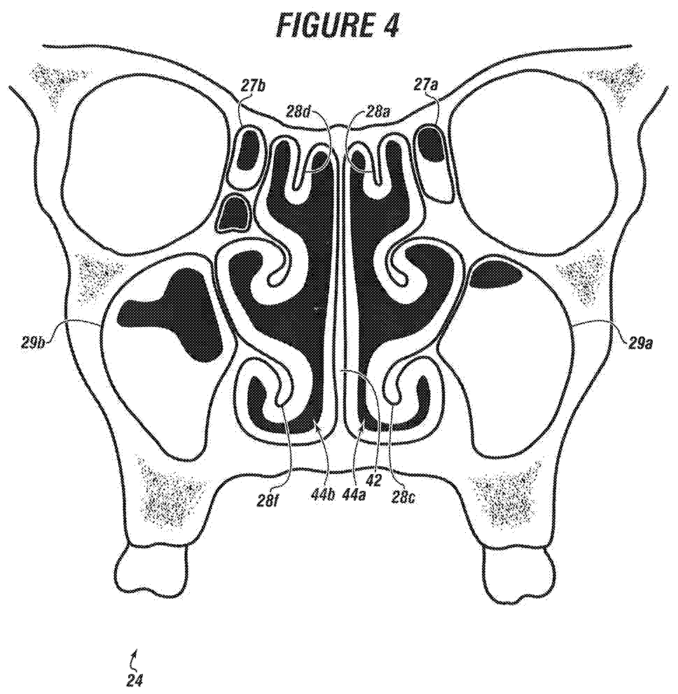

FIG. 4 depicts a computer tomography scan 24 of congested nasal and sinus cavities according to one or more embodiments.

In embodiments, the patient can have at last one turbinate 28a-28f, at least one maxillary sinus 29a and 29b, a pair of ethmoid sinuses 27a and 27b, a nasal septum 42, and nasal cavities 44a and 44b.

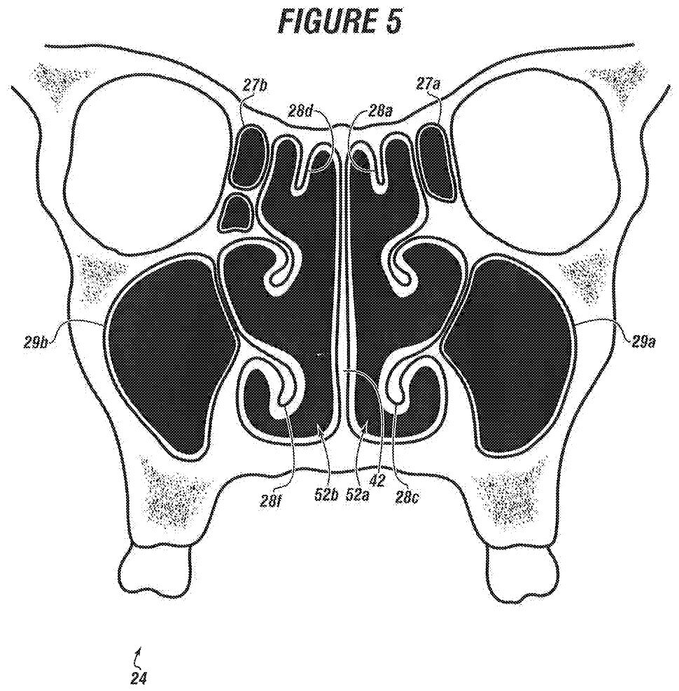

FIG. 5 depicts a computer tomography scan of anesthetized decongested nasal cavities according to one or more embodiments.

The computer tomography scan 24 of anesthetized decongested nasal cavities 52a and 52b are shown.

In embodiments, the computer tomography scan can show the at least one turbinate 28a-28f, the at least one maxillary sinus 29a and 29b, the pair of ethmoid sinuses 27a and 27b, and the nasal septum 42.

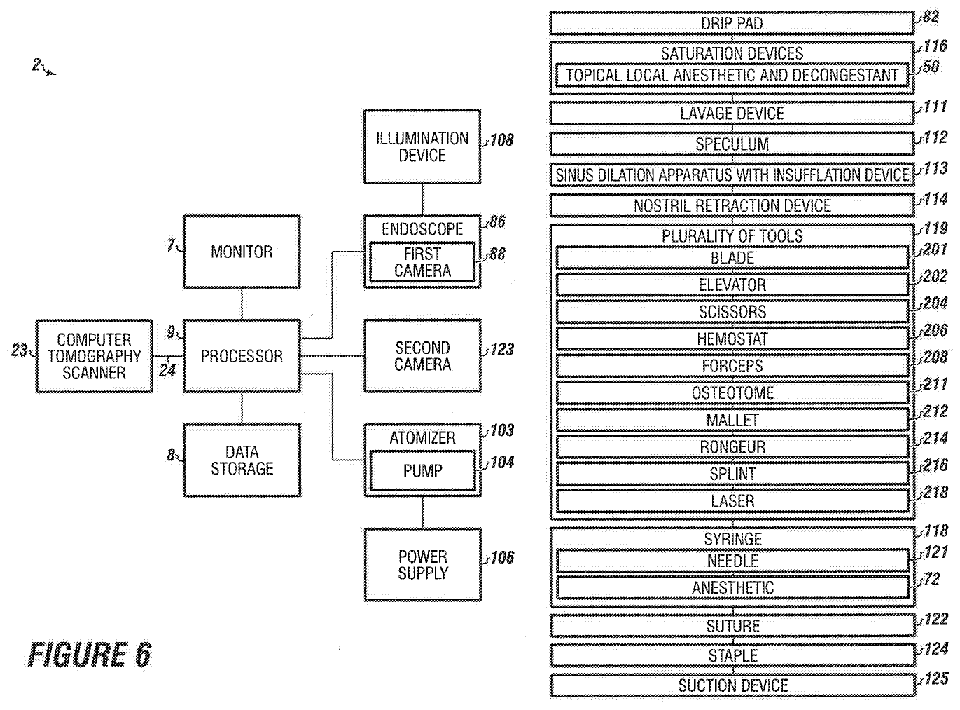

FIG. 6 depicts equipment used to implement migraine reduction according to one or more embodiments.

The equipment for treating a headache via at least one nostril of a patient, includes a computer tomography scanner 23 (CT scanner) configured to perform the computer tomography scan 24 on at least one sinus cavity of the patient.

In embodiments, the equipment may include a processor 9, a data storage 8 and a monitor 7.

In embodiments, the kit 2 can contain an atomizer 103 with a pump 104, which can be connected to a power supply 106.

For example, the atomizer can be a hand atomizer, a spray bottle, a steam generating device, or a humidifier. In embodiments, the power supply can be 110 AC.

In embodiments, the atomizer 103 can be used for spraying a nasal cavity of the patient with a topical local anesthetic and decongestant 50 to form an anesthetized decongested nasal cavity in the patient.

An illumination device 108, such as a light emitting diode (LED) with a power supply, can be configured to: (i) enable inspection of the anesthetized decongested nasal cavity to confirm that the proposed medical treatment plan to implement is the best match and (ii) enable inspection between mucosal flaps for a residual septal bone fragment, a residual fragment of septal cartilage, or combinations thereof.

In embodiments, the illumination device can be used in conjunction with a monitor, light cord, endoscope and a camera to facilitate activity to close the mucosal flaps, sew with suture, staple an incision, dilate the sinus cavities, suction the anesthetized decongested nasal cavity, treat turbinates, or other surgical procedure on the anesthetized decongested nasal cavity.

In embodiments, a sinus ostium dilation apparatus with insufflation device 113 can be configured to perform dilation of at least one sinus ostium using the proposed medical treatment plan with the greatest number of matches.

In embodiments, the illumination device 108 can be mounted to an endoscope 86. The endoscope 86 can have a first camera 88 for providing images to the data storage 8 connected to the processor 9, which can be in further communication with the monitor 7.

In embodiments, a second camera 123 can be configured to take a still image, a video image, or both a still image and a video image prior to therapy or post therapy, and store the image or images in the data storage 8.

In embodiments, the equipment may include a drip pad 82, which can be positioned adjacent to at least one nostril of the patient.

In embodiments, the equipment may include a plurality of saturation devices 116, such as cottonoids. Each saturation device 116 can be saturated with a topical local anesthetic and decongestant 50.

In embodiments, each saturation device 116 can be used to contact at least one turbinate of the patient to decongest the at least one turbinate.

In embodiments, a lavage device 111 can be used for performing a lavage after removing at least partially or totally, at least one ethmoid sinus of the patient or dilating the ostium of a frontal, maxillary or sphenoid sinus of the patient.

In embodiments, a speculum 112 and a nostril retraction device 114 can be configured to expose at least one mucosal flap of the nasal septum.

In embodiments, the sinus dilation apparatus with insufflation device 113 can be configured to perform dilation on all sinus ostia except ethmoid sinuses of the patient.

In embodiments, a syringe 118 with a needle 121 can be used to infuse an anesthetic 72 into the nasal cavity of the patient. For example, from 10 cc to 40 cc of anesthetic can be used on an average adult of 75 kilograms.

In embodiments, the equipment may include a plurality of tools 119. In embodiments, the plurality of tools may include but is not limited to: a blade 201, an elevator 202, a scissors 204, a hemostat 206, forceps 208, an osteotome 211, a mallet 212, a rongeur 214, a splint 216, and a laser 218.

In embodiments, the scissors 204 can be double action scissors or turbinate scissors.

In embodiments, the forceps can be bayonet forceps.

A portion of the plurality of tools can remove bone and cartilage of the nasal septum, reconstruct cartilage of the nasal septum, reposition at least one of: bone and cartilage of the nasal septum, at least partially remove at least one ethmoid sinus of the patient, and open at least one maxillary sinus ostium of the patient.

In embodiments, some of the plurality of tools can be configured to perform at least one operation on the cartilage, the bone or both the cartilage and the bone, comprising: chiseling, cutting, fracturing, incising, and shaving.

In embodiments, the laser 218 can be used to burn at least one turbinate. In embodiments, the equipment may include a suture 122 or at least one staple 124 to surgically close the exposed mucosal flaps of the nasal septum.

In embodiments, the equipment may include a suction device 125 to remove unwanted matter from the anesthetized decongested nasal cavity.

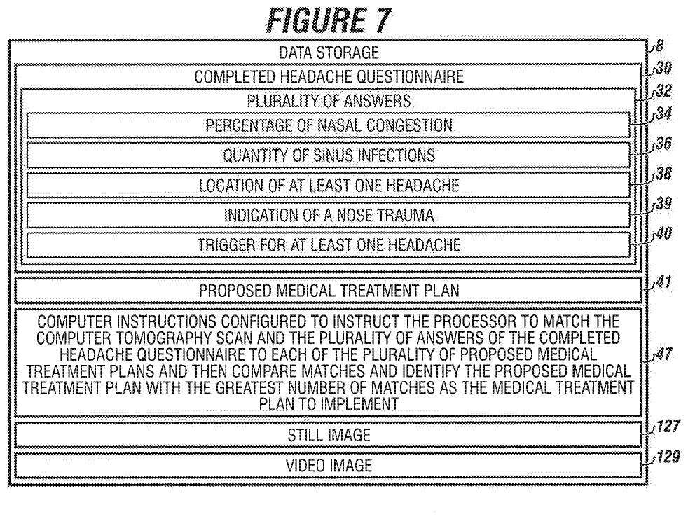

FIG. 7 depicts a data storage according to one or more embodiments.

The term “data storage” refers to a non-transitory computer readable medium, such as a hard disk drive, solid state drive, flash drive, tape drive, and the like. The term “non-transitory computer readable medium” excludes any transitory signals but includes any non-transitory data storage circuitry, e.g., buffers, cache, and queues, within transceivers of transitory signals.

In embodiments, the data storage 8 can contain a headache questionnaire, a plurality of answers 32 from the completed headache questionnaire 30, a still image 127 and a video image 129.

The plurality of answers 32 can indicate a percentage of nasal congestion 34, such as a range from 0.1 percent to 100 percent, a quantity of sinus infections 36, such as the number experienced by the patient during a preset unit of time, a location of at least one headache 38, an indication of a nose trauma 39, and a trigger for the at least one headache 40.

In embodiments, the data storage 8 can contain a plurality of proposed nasal/sinus surgical treatment plans 41. In embodiments, the data storage 8 may include computer instructions 47 configured to instruct a processor to match the computer tomography scan and the plurality of answers of the completed headache questionnaire to each of the plurality of proposed nasal/sinus surgical treatment plans and then compare the matches to identify the proposed nasal/medical surgical treatment plan with the greatest number of matches as the nasal/sinus surgical treatment plan to implement.

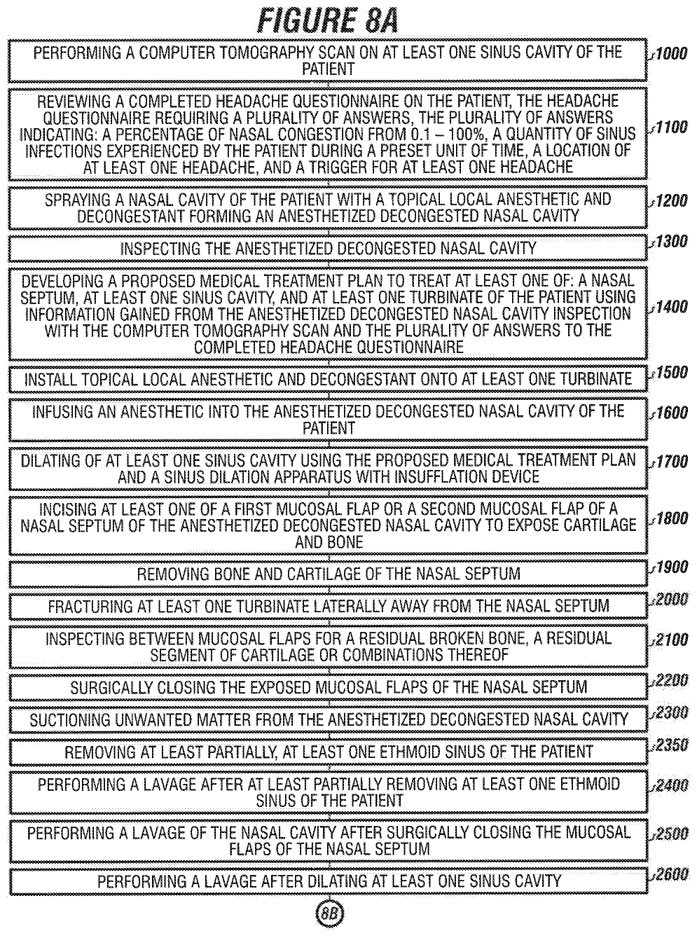

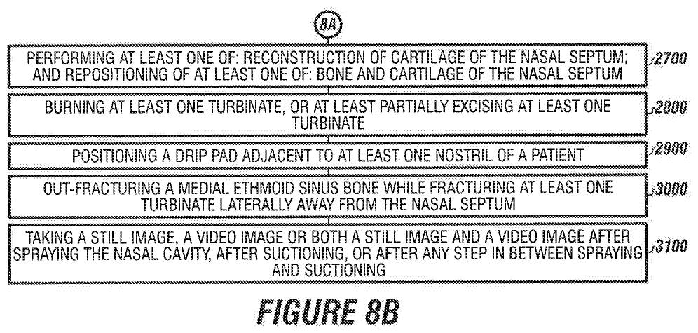

FIG. 8 depicts a method to use the kit to reduce migraine headaches in a patient according to one or more embodiments.

In embodiments, the method when used with the kit (should . . . and equipment . . . be added here like in 00135?) can reduce the frequency and intensity of headaches, such as migraines, in a patient by at least 50 percent.

Further, in embodiments, the method when used with the kit and equipment can reduce headaches, such as migraines, in a patient population by at least 88 percent.

The method for treating a headache, such as a migraine, via at least one nostril of a patient may include performing a computer tomography scan on the nasal cavity and at least one sinus cavity of the patient, as shown in box 1000. The method may include reviewing a completed headache questionnaire on the patient or acquiring patient data, the completed headache questionnaire requiring a plurality of answers, the plurality of answers indicating a percentage of nasal congestion from 0.1 percent to 100 percent, a quantity of sinus infections experienced by the patient during a preset unit of time, a location of at least one headache, and a trigger for at least one headache, as shown in box 1100.

For example, the system may use the following stored data collected from the patient:
How long the patient has suffered from headaches (weeks, months, years)
Frequency (number per week or month, standardized to number per month for analysis)
Location (Typical location for rhinogenic headaches include: forehead, temples, periorbital, cheek, top of head, perinasal)
Severity of pain (minimum, maximum, and average, on a scale of 1 to 10)
Quality of pain (e.g., constant, pounding, etc.)
Time of day or night of pain onset
Triggers that cause the headaches
Occurrence of sinus infections
Occurrence of hypertension
Family history of headaches
History of nasal or facial trauma
Amount of nasal congestion from 0.1 percent to 100 percent
Previous treatment history for headaches

In embodiments, the plurality of answers may include an indication of a nose trauma.

In embodiments, the plurality of answers can be stored in the data storage connected to the processor using an input device connected to the processor, wherein the input device can be a human machine interface, such as a mobile phone application or a computer application.

The method may include spraying a nasal cavity of the patient with a topical local anesthetic and decongestant forming an anesthetized decongested nasal cavity, as shown in box 1200.

The topical anesthetic and decongestant can be a formulation having 50 weight percent to 80 weight percent of a phenylepinephrine and 20 weight percent to 50 weight percent of at least one of: a lidocaine, oxymethazoline, and a tetracaine.

In embodiments, additional components can be added to the formulation and be usable herein. Some of the additional components can be at least one of: 1 weight percent to 5 weight percent of a peppermint oil, 1 weight percent to 5 weight percent of a menthol, 1 weight percent to 10 weight percent of an emu oil, 1 weight percent to 5 weight percent of an eucalyptus oil, 1 weight percent to 5 weight percent of a lemon oil, 1 weight percent to 5 weight percent of a rosemary oil, 1 weight percent to 5 weight percent of a tea tree oil, 1 weight percent to 5 weight percent of a pine oil, 1 weight percent to 5 weight percent of a lavender oil, 1 weight percent to 5 weight percent of a thyme oil, and 1 weight percent to 5 weight percent of a camphor.
(i) Formulation Example 1:

As an example, the formulation can be 60 weight percent of phenylephrine, 30 weight percent of a lidocaine, 3 weight percent of peppermint oil, 4.5 weight percent of emu oil, and 2.5 weight percent of lemon oil.
(j) Formulation Example 2:

As an example, the formulation can be 68 weight percent of phenylephrine, 20 weight percent of a tetracaine, 2 weight percent of menthol, 5 weight percent of tea tree oil, and 4 weight percent of pine oil and 1 weight percent of camphor.
(k) Formulation Example 3:

As an example, the formulation can be 40 weight percent of phenylephrine, 50 weight percent of oxymetazoline, 3.5 weight percent of eucalyptus oil, 1.5 weight percent of rosemary oil, 3 weight percent of lavender oil, and 2 weight percent of thyme oil.

In embodiments, the topical local anesthetic and decongestant may include from 10 weight percent to 20 weight percent of a nasal steroid, such as triamcinolone acetonide.
(l) Formulation Example 4:

As an example, the formulation can be 60 weight percent of phenylephrine, 30 weight percent of a lidocaine and 10 weight percent of triamcinolone acetonide.

The method may include inspecting the anesthetized decongested nasal cavity, as shown in box 1300.

The method may include using an algorithm to match a proposed medical treatment plan to treat at least one of: a nasal septum, at least one sinus cavity, and at least one turbinate of the patient with the computer tomography scan and the plurality of answers to the completed headache questionnaire, as shown in box 1400.

The method may include installing topical local anesthetic and decongestant onto at least one turbinate, as shown in box 1500.

The method may include infusing an anesthetic into the anesthetized decongested nasal cavity of the patient, as shown box 1600.

In embodiments, the anesthetic is not a topical anesthetic. For example, the anesthetic can be injectable lidocaine HCL 1 weight percent with epinephrine 1:100,000, such as for infiltration and nerve block.

The method may include dilating of at least one sinus ostium using the proposed medical treatment plan and a sinus dilation apparatus with insufflation device, as shown box 1700.

In embodiments, the dilating can be with a balloon or another non-balloon method.

In embodiments, sponges or splints can be used to stabilize the nasal cavity.

The method may include incising at least one of: a first mucosal flap or a second mucosal flap of a nasal septum of the anesthetized decongested nasal cavity to expose bone, cartilage or bone and cartilage, as shown in box 1800.

The method may include removing bone, cartilage or bone and cartilage of the nasal septum, as shown in box 1900.

The method can involve fracturing at least one turbinate laterally away from the nasal septum, as shown in box 2000.

the method can involve inspecting between mucosal flaps for a residual septal bone fragment, residual fragment of cartilage, or combinations thereof, as shown in box 2100.

The method can involve surgically closing the exposed mucosal flaps of the nasal septum, as shown in box 2200.

The method can involve suctioning unwanted matter from the anesthetized decongested nasal cavity, as shown in box 2300.

The method can involve removing at least partially, at least one ethmoid sinus of the patient, as shown in box 2350.

The method can involve performing an ethmoid lavage after at least partially removing at least one ethmoid sinus of the patient, as shown in box 2400.

The method can involve performing a lavage of the nasal cavity after surgically closing the mucosal flaps of the nasal septum as shown in box 2500.

The method can involve performing a lavage after dilating at least one sinus ostium, as shown in box 2600.

The method can involve performing at least one of: reconstruction of cartilage of the nasal septum and repositioning of at least one of: bone and cartilage of the nasal septum, as shown in box 2700.

The method can involve burning at least one turbinate, or at least partially excising at least one turbinate, as shown in box 2800.

In embodiments, any one of the following steps of the method can be performed and in no specific order:

The method may include positioning a drip pad underneath at least one nostril of the patient, as shown in box 2900.

The method may include out-fracturing a medial ethmoid sinus bone while fracturing at least one middle turbinate laterally away from the nasal septum, as shown in box 3000.

The method may include taking a still image, a video image or both a still image and a video image after spraying the nasal cavity, after suctioning, or after any step in between spraying and suctioning, as shown in box 3100.

In embodiments, the dilating can be performed on any number of sinus ostium in a patient, such as at least one sinus ostium or all sinus ostia of the patient.

In embodiments, the reconstruction of cartilage can involve performing at least one of: suturing, cutting, fracturing, incising, and shaving.

In embodiments, the inspecting of the anesthetized decongested nasal cavity can be with a configured illumination device.

In embodiments, the illumination device can be mounted to an endoscope. In embodiments, the endoscope can be attached to a camera that can connect to a monitor.

In embodiments, the unwanted matter can be a member of the group: mucus, blood, pus, irrigation fluid, and combinations thereof.

As an example, a patient, Kelvin has debilitating migraine headaches that require him to stay home from work and lay down in a dark room for several hours.

To heal Kelvin, the following steps are implemented.

First, Kelvin is given an initial consult to determine the extent of his headaches, problems in his head, and the root cause of his migraine headache.

In the initial consultation, Kelvin is provided with the headache questionnaire and asked to provide a plurality of answers.

Then Kelvin has his nasal cavity sprayed with a sufficient amount of a topical local anesthetic and decongestant, such as a formulation of 50 weight percent of phenylephrine and a 50 weight percent of lidocaine to coat all the nasal cavities. About 0.5 cubic centimeters to 2 cubic centimeters of the topical local anesthetic and decongestant is used for Kelvin.

An illumination device is used with an endoscope having a camera to inspect the nose. A proposed medical treatment plan is tentatively developed to treat nasal/sinus abnormalities.

Kelvin obtains a computer tomography scan to confirm the diagnosis developed from the inspection.

The resulting computer tomography scan is reviewed, and Kelvin is diagnosed with having chronic sinusitis in addition to a deviated nasal septum with enlarged turbinates.

The proposed medical treatment plan is revised in view of the review of the computer tomography scan. A surgical procedure date is planned with the patient.

On the day of surgery, a topical local anesthetic and decongestant is applied onto the enlarged turbinates with a waiting period from 2 minutes to 5 minutes.

An anesthetic is injected with syringe and needle into the nasal septum and turbinates.

All of Kelvin's sinus ostia except for ethmoid sinuses are dilated with an ACCLARENT™ balloon sinuplasty apparatus, which takes from approximately 8 minutes to 15 minutes.

One of the mucosal flaps is incised to expose cartilage and bone and a speculum is used to retract the flap.

Offending cartilage and bone, which forms the deviated nasal septum is removed. An inspection is made between the mucosal flaps for fractured bone and cartilage segments. Suctioning or removal of bone fragments or cartilage segments is performed. The mucosal flap is surgically closed. Additional suctioning is performed to remove unwanted matter. At least one turbinate is then outfractured laterally away from the nasal septum. Kelvin experiences a full elimination in migraine headache occurrences.

While these embodiments have been described with emphasis on the embodiments, it should be understood that within the scope of the appended claims, the embodiments might be practiced other than as specifically described herein.

What is claimed is:

1. A method for selecting a surgical headache treatment plan to treat at least one headache and/or migraine of a patient by performing nasal/sinus surgery, the method comprising:
a. performing a computer tomography scan of a nasal cavity of the patient;
b. performing a computer tomography scan of a sinus cavity of the patient;
c. reviewing the results of the computer tomography scan of the nasal cavity and of the sinus cavity to identify one or more datum showing one or more structural abnormality from the group comprising: nostril asymmetry, nasal bone fracture, deviated crista galli, deviated septal cartilage or bone, septal spur, enlarged turbinate, turbinate concha bullosa, contact between nasal structures, nasal polyps, nasal scars, narrow sinus ostium, inflamed sinus, obstructed sinus ostium, narrow frontal sinus recess, obstructed frontal sinus recess, thick sinus mucosa, sinus infection, sinus cyst, sinus polyp, sinus osteoma and sinus opacification;
d. reviewing a set of headache and congestion data provided by the patient for two or more datum indicating at least two of the following:
(i) an amount of nasal congestion experienced by the patient;
(ii) a quantity of sinus infections experienced by the patient;
(iii) a location of headache pain;
(iv) a severity of headache pain;
(v) a duration of headache pain;
(vi) a frequency of headache pain;
(vii) a quality of pain;
(viii) a trigger for the at least one headache;
(ix) a length of time the patient has suffered from headaches;
(x) a frequency of headache occurrence;
(xi) a time of day for which the headaches can appear;
(xii) an occurrence of hypertension;
(xiii) a family history of headaches;
(xiv) a type of nasal or facial trauma;
(xv) a prior diagnosis of migraine;
(xvi) a type of prior migraine diagnosis;
(xvii) a prior treatment of headaches; and
(xviii) a symptom associated with headache pain;
(xix) a lifestyle impact associated with headache pain; and
e. matching the computer tomography scan data and the patient's headache data to a surgical headache treatment plan comprising one or more nasal/sinus procedures to be performed on the patient selected from the group of procedures comprising: septal bone fracture, septal cartilage fracture, septal bone shaving, septal cartilage shaving, septal bone removal, septal cartilage removal, turbinate fracture, turbinate ablation, turbinate reduction, turbinate cautery, partial turbinectomy, nasal scar lysis, nasal polyp removal, sinus ostium enlargement, sinus lavage, sinus polyp removal, sinus cyst removal, sinus osteoma removal, sinus mucosa removal, sinus bone removal, and sinus pus removal.

2. A method as claimed in claim 3, wherein the selected headache treatment plan, when successfully conducted, reduces by 50% at least one headache characteristic experienced by the patient as reported by the patient selected from the group comprising: frequency and intensity.

3. A method as claimed in claim 3 wherein, in the matching step, (a) a computer tomography scan data showing a deviation of the septum with a maxillary crest causing nasal impairment and (b) patient headache data indicating at least one condition selected from the group comprising: (1) recurrent nasal congestion, (2) recurrent headache and (3) migraine is matched with (c) at least one surgical headache treatment plan selected from the group comprising: (1) removal of the maxillary crest with an osteotome; (2) endoscopic removal of the maxillary crest for an isolated abnormality; and (3) removal of the maxillary crest cartilage displacement with an instrument.

4. A method as claimed in claim 3 wherein, in the matching step, (a) a computer tomography scan data showing a mild to moderate deviation of the septal cartilage and (b) patient headache data indicating mild to moderate nasal impairment in breathing with instances of recurring headache or migraine is matched with (c) a surgical headache treatment plan selected from the group comprising performing: (1) a closed fracture of the septum, (2) an endoscopic removal of the deviation, and (3) a repair closure of the septal mucosal flaps.

5. A method as claimed in claim 3 wherein, in the matching step, (a) a computer tomography scan data showing a severe deviation of the septal cartilage and (b) patient headache data showing severe nasal impairment in breathing with instances of recurring headache or migraine is matched with (c) at least one surgical headache treatment plan selected from the group comprising: (1) removing the severe deviation of the septal cartilage while attempting to leave behind cartilage dorsally and caudally to maintain nasal tip and dorsal support, (2) reconstructing the septal cartilage with a straight piece of septal cartilage and (3) performing a repair closure of the septal mucosal flaps.

6. A method as claimed in claim 3 wherein, in the matching step, (a) a computer tomography scan data showing a moderate to severe caudal cartilage deviation and nostril asymmetry and (b) patient headache data showing moderate to severe nasal impairment in breathing with recurring headaches or migraines is matched with (c) at least one surgical headache treatment plan selected from the group comprising: (1) shaving the deviated caudal septum up to 2 mm, (2) removing and reconstructing the deviated cartilage with a straight piece of septal cartilage, (3) performing a repair closure of the septal mucosal flaps with dissolvable suture, and (4) splinting the repair

7. A method as claimed in claim 3 wherein, in the matching step, (a) a computer tomography scan data showing a mild deviation of the bony septum in contact with a turbinate and (b) patient headache data showing mild breathing impairment and recurring headache or migraine is matched with (c) at least one surgical headache treatment plan selected from the group comprising: (1) fracturing the deviation to the midline, (2) removing the deviation endoscopically or with traditional septoplasty, based upon which one or more of these procedures will result in most removal of any bone that can contact the turbinate, and (3) performing a repair closure of a septal mucosal flap.

8. A method as claimed in claim 3 wherein, in the matching step, (a) a computer tomography scan data showing a moderate to severe deviation of the bony septum with contact with a turbinate and (b) patient data indicating moderate to severe impaired breathing with recurrent headache or migraine is matched with (c) at least one surgical headache treatment plan selected from the group comprising: (1) fracturing the deviation to the midline, (2) correcting the deviation endoscopically, (3) correcting the deviation with traditional septoplasty (4) otherwise removing bone approximate to the turbinate, (4) performing a repair closure of the septal mucosal flaps.

9. A method as claimed in claim 3 wherein, in the matching step, (a) a computer tomography scan data showing one or more isolated septal spur and (b) patient data showing no nasal breathing impairment and recurrent headaches or migraine is matched with (c) at least one surgical headache treatment plan selected from the group comprising: (1) endoscopic removal the one or more isolated spur, (2) open septoplasty to remove the one or more isolated spurs and (3) repair closure of the septal mucosal flaps with dissolvable suture.

10. A method as claimed in claim 3 wherein, in the matching step, (a) a computer tomography scan data showing at a condition of the patient's frontal sinus selected from the following group of conditions: (1) a narrow ostium, (2) an obstructed ostium, (3) mucosal thickening, and (4) opacification and (b) patient data indicating recurrent pressure headache in the forehead is matched with (c) a surgical headache treatment plan selected from the group comprising: (1) frontal balloon sinuplasty with lavage and (2) removal of the frontal recess cells.

11. A method as claimed in claim 3 wherein, in the matching step, (a) a computer tomography scan data showing osteoma of the frontal sinus and (b) patient data indicating recurrent pressure headache in the forehead is matched with (c) a headache treatment plan for (1) removing the osteoma if the osteoma is located in the recess cells and (2) removing any higher osteoma only if headache symptoms persist in the forehead after removal of all lower osteoma.

12. A method as claimed in claim 3 wherein, in the matching step, (a) a computer tomography scan showing at least one condition of the maxillary sinus selected from the group comprising: (1) opacification, (2) mucosal thickening, (3) ostium, (4) a narrow outflow tract; or (4) an obstructed outflow tract; and (b) patient data indicating at least one headache condition from the group comprising: (1) pressure headache in the cheek, (2) pain under the eye, and (3) tooth pain is matched with (c) at least one surgical headache treatment plan from the group comprising: (1) maxillary balloon sinuplasty and (2) maxillary antrostomy.

13. A method as claimed in claim 3 wherein, in the matching step, (a) a computer tomography scan showing at least one moderately sized cyst in the maxillary sinus and (b) patient data indicating pain or pressure headache in the cheek or under the eye or tooth pain is matched with (c) a surgical headache treatment plan selected to remove the cyst from the group comprising: (1) balloon sinuplasty to dilate the maxillary sinus ostium to insert an instrument(s) to remove the cyst; (2) an traditional endoscopic sinus technique using forceps or tool made for rupturing; and (3) removing the cyst with lavage.

14. A method as claimed in claim 3 wherein, in the matching step, (a) a computer tomography scan showing a condition of the sphenoid sinus selected from the group of conditions comprising: (1) opacification, (2) mucosal thickening, (2) narrow or obstructed outflow tract, (4) ostium and (b) patient data indicating pain or pressure headache at the top of the head is matched with (c) a surgical headache treatment plan selected from the group comprising: (1) sphenoid balloon sinuplasty with lavage and (2) traditional endoscopic sphenoid sinuplasty.

15. A method as claimed in claim 3 wherein, in the matching step, (a) a computer tomography scan showing anterior opacification, mucosal thickening or osteoma of the ethmoid sinus and (b) patient data indicating pain or headache between or behind the eyes is matched with (c) anterior ethmoidectomy.

16. A method as claimed in claim 3 wherein, in the matching step, (a) a computer tomography scan showing one condition from the group of conditions of the ethmoid sinus selected from (1) complete opacification; (2) mucosal thickening of the total or posterior thereof; (3) osteoma in the posterior, and (b) patient data indicating pain or headache between or behind the eyes is matched with (c) a headache treatment plan comprising an ethmoidectomy.

17. A method as claimed in claim 3 wherein, in the matching step, (a) a computer tomography scan data showing inferior turbinate contact with the septum and (b) patient data indicating no nasal airway impairment is matched with (b) a surgical treatment plan to eliminate said interior turbinate contract selected from the group comprising: (1) outfracturing the inferior turbinate away from the septum and (2) otherwise eliminating the contact.

18. A method as claimed in claim 3 wherein, in the matching step, (a) a computer tomography scan showing mild enlargement of an inferior turbinate in contact with the septum and (b) patient data indicating nasal airway impairment experienced by the patient less than or equal to 50% of the time is matched with (c) at least one headache treatment plan selected from the group comprising: (1) out-fracturing the contacting inferior turbinate away from the septum and (2) ablating turbinate tissue.

19. A method as claimed in claim 3 wherein, in the matching step, (a) a computer tomography scan data showing at least one moderately to severely enlarged inferior turbinate in contact with the septum and (b) patient data indicating nasal airway impairment experience by the patient more than 50% of the time is matched with (c) at least one surgical treatment plan to eliminate the contact selected from the group comprising: (1) outfracturing the contacting inferior turbinate away from the septum and (2) excising the contacting inferior turbinate and cauterizing the edges of the remnant thereof.

20. A method as claimed in claim 3 wherein, in the matching step, (a) a computer tomography scan showing the patient's middle turbinates to have mild hypertrophy with approximation to the septum and (b) patient data indicating perinasal pain or headache between the eyes is matched with (c) a surgical headache treatment plan to reduce their width.

21. A method as claimed in claim 3 wherein, in the matching step, (a) a computer tomography scan data showing the patient's middle turbinates to have moderate hypertrophy with close approximation to or contact with the septum and (b) patient data indicating perinasal pain or headache between the eyes is matched with (c) a surgical headache treatment plan selected from the group comprising: (1) reducing said approximation to or contact with the septum by partially excising the inferior 4-8 mm margin and (2) out-fracturing them away from the septum.

22. A method as claimed in claim 3 wherein, in the matching step, (a) a computer tomography scan showing the patient's middle turbinate has large hypertrophy with contact with the septum and (b) patient data indicating perinasal pain or headache between the eyes is matched with (c) a surgical headache treatment plan to reduce the size of the contacting turbinate selected from the group comprising: (1) partially excising an inferior 4-8 margin of the turbinate and cauterizing the remnant and (2) otherwise surgically reducing the size of the turbinate.

23. A method as claimed in claim 3 wherein, in the matching step, (a) a computer tomography scan data showing a small sized concha bullosa of the middle turbinate and (2) patient data indicating at least one of perinasal pain and headache between the eyes is matched with (b) a surgical headache treatment plan selected to reduce the width of the concha bullosa selected from the group comprising: (1) squeezing the concha bullosa with forceps and (2) otherwise operating on the patient to reduce the width of the concha bullosa.

24. A method as claimed in claim 3 wherein, in the matching step, (a) a computer tomography scan data showing one of a medium or large sized concha bullosa of the middle turbinate and (b) patient data indicating at least one of perinasal pain and headache between the eyes is matched with (c) a surgical headache treatment plan to reduce the size of the concha bullosa selected from the group comprising: (1) excising the lateral portion of the concha bullosa and cauterizing the edges of the remnant; (2) otherwise operating on the patient to reduce the size of the concha bullosa.

25. A method as claimed in claim 3 wherein, in the matching step, (a) a computer tomography scan data showing at least one of the patient's superior and supreme turbinates in contact with the septum and (b) patient data indicating recurrent headaches is matched with (c) a surgical headache treatment plan to eliminate the touching between the contacting turbinate and the septum selected from the group comprising: (1) outfracturing the contacting turbinate away from the septum; (2) otherwise eliminating contact between the contacting turbinate and septum.
